(19) United States
(12) Patent Application Publication
(10) Pub. No.: US 2021/0137747 A1
(43) Pub. Date: May 13, 2021

(54) WASHABLE MULTILAYER NURSING PAD

(71) Applicant: CACHE-COEUR, Le Relecq Kerhuon (FR)

(72) Inventor: Audrey TROLLIET, Brest (FR)

(21) Appl. No.: 16/623,598

(22) PCT Filed: Jun. 25, 2018
(86) PCT No.: PCT/FR2018/051531

§ 371 Date: Dec. 17, 2019

(30) Foreign Application Priority Data
Jun. 23, 2017 (FR) 1755791

(51) Int. Cl. A61F 13/14; A61F 13/514; A61F 13/511; A61F 13/512; A61F 13/15

(52) U.S. Cl. CPC A61F 13/141; A61F 13/5148; A61F 13/5116; A61F 13/512; A61F 2013/53062; A61F 13/15699; A61F 2013/15016; A61F 2013/51139; A61F 13/51113

(57) ABSTRACT

A washable multilayer nursing pad, including an edge and successive layers: a knitted mesh layer at least 1.5 mm thick and perforated, including a top face in direct contact with the skin and a bottom face; a layer of non-woven fibres, in contact with the top face, capable of absorbing at least 2.5 L/m2 of water; a microporous layer including an impermeable and breathable structure; and a protective layer covering the entire microporous layer.

### CROSS REFERENCE TO RELATED APPLICATIONS

This application is a National Stage of International Application No. PCT/FR2018/051531, having an International Filing Date of 25 Jun. 2018, which designated the United States of America, and which International Application was published under PCT Article 21(2) as WO Publication No. 2018/234719 A1, which claims priority from and the benefit of French Patent Application No. 1755791, filed on 23 Jun. 2017, the disclosures of which are incorporated herein by reference in their entireties.

### BACKGROUND 1. FIELD

The present disclosure relates to a washable multilayer nursing pad. It applies, in particular, to the field of underwear for mothers-to-be or nursing mothers having the need to absorb breast milk discharge.

### 2. BRIEF DESCRIPTION OF RELATED DEVELOPMENTS

Two types of pads are known: disposable and washable. A washable nursing pad is a reusable and long-lasting pad unlike disposable nursing pads.

Disposable nursing pads may cause irritations due to the plastic film used for imperviousness which forms a barrier to air circulation. Furthermore, disposable pads are composed of toxic substances and are single-use.

Existing washable pads do not separate the skin from moisture, which causes irritations and sticks the materials to the skin.

Washable pads are flat, those which are not take shape with a gripper which creates leakage due to the needle holes. Finally, washable pads offer an average absorption capacity of 20 ml of breast milk per pad and disposable pads offer a maximum absorption of 75 ml.

Some prior art documents also propose washable nursing pad devices. There is known for example the publication document WO2005034824 wherein is described a pad which comprises three layers sealed together at the periphery thereof by ultrasonic sealing. However, this type of pad is not satisfactory as it does not separate the skin from moisture and is not antibacterial. This pad is not satisfactory as the materials are free and the inner layer can stick to moist skin. This pad is not ergonomic and the shape thereof is not suitable for different shapes of bras.

### SUMMARY

The aim of the present disclosure is that of remedying these drawbacks.

For this purpose, according to a first aspect, the present disclosure relates to a washable multilayer nursing pad, characterised in that it comprises an edge and successive layers:

a knitted mesh layer at least 1.5 mm thick and perforated, comprising a top face in direct contact with the skin and a bottom face;

a non-woven fibre layer, in contact with the top face, capable of absorbing at least 2.5 L/m2 of water;

a microporous layer comprising an impermeable and breathable structure;

a protective mesh layer covering the entire microporous layer.

The knitted mesh layer is three-dimensional (3D) and enables air circulation and the skin to be kept dry by separating it from moisture. The 3D knitted mesh layer makes it possible to prevent bacterial proliferation. The 3D knitted mesh layer makes it possible to prevent any odours.

The knitted mesh layer comprises an aerated core.

Concerning the thickness of 1.5 mm. This thickness is measured as per the ISO 5084 standard. The thickness measurer adopts the principle of determining the thickness of a textile which consists of measuring the distance perpendicularly between two reference sheets, herein between the top face and bottom face of the knitted mesh layer, applying a pressure of 0.1 kPa or 1 kPa on the textile.

The interlocking of the non-woven fibres is mechanical. The layer of non-woven fibres is produced using needles which convert the fibres into a non-woven material. The material forming the layer of non-woven fibres lock the liquid after absorption. The layer of non-woven fibres is superabsorbent.

Washable nursing pads according to the prior art offer an average absorption of 20 ml of breast milk per pad. Disposable nursing pads offer an average absorption of 75 ml of breast milk. The layer of non-woven fibres provides the washable pad as described with an absorption capacity at least equal to disposable nursing pads. The absorption capacity is at least 65 ml of breast milk, ranging up to 80 ml of breast milk.

The membrane forms a barrier to the liquid while allowing air to circulate towards the skin accelerating drying. The microporous membrane makes it possible to prevent breast milk leakages. The membrane is covered with a protective mesh layer.

The disclosure is advantageously implemented according to the aspects of the disclosure and alternative aspects of the disclosure described hereinafter, which are to be considered individually or according to any technically effective combination.

In an aspect of the disclosure, the knitted mesh layer comprises:

the top face composed of a first textile fibre layer in direct contact with the skin, said first textile fibre layer is composed of a plurality of perforating openings on an outer surface;

the bottom face composed of a second textile fibre layer in contact with the non-woven fibre layer;

a monofilament layer at least 1.5 mm thick composed of textile fibre, inserted between the first textile fibre layer and the second textile fibre layer.

The term textile fibre denotes a natural, synthetic or artificial fibre.

In an aspect of the disclosure, the first textile fibre layer is made of antibacterial polyester and the second fibre layer is made of polyester.

According to the two preceding aspects of the disclosure, the knitted mesh layer is composed of three layers of three different materials and comprises:

the top face composed of an antibacterial polyester layer, said antibacterial polyester layer is composed of a plurality of perforating openings on an outer surface;

the bottom face composed of a polyester layer in contact with the non-woven fibre layer;

a knitted monofilament layer at least 1.5 mm thick composed of polyester, between the antibacterial polyester layer and the polyester layer.

The antibacterial fibre makes it possible to prevent any bacterial proliferation caused by breast milk proteins for guaranteed hygiene.

The perforated structure enables breast milk drainage. The perforations have an opening of a size of the order of magnitude of a needle.

The very aerated 3D structure of the monofilament layer enables air circulation. The very aerated structure of the monofilament layer separates the skin from moisture. The structure of the monofilament layer reduces the risk of irritation considerably.

In an aspect of the disclosure, the non-woven fibre layer has a thickness between 2.5 mm and 8 mm, preferably between 5 mm and 7 mm.

The greater the thickness of the non-woven fibre layer, the higher the absorption capacity of the pad.

In an aspect of the disclosure, the pad comprises a triangular shape with rounded angles.

The triangular shape makes it possible to render the pad invisible under clothing. The triangular shape is suitable for all shapes of bra.

In an aspect of the disclosure, the pad has two sizes suitable for varying the absorption capacity according to the flows and adapting to different bust volumes.

In an aspect of the disclosure, the pad comprises a rounded preformation to mould the shape of the breast.

The preformation to mould the shape of the breast makes it possible to prevent a sensation of discomfort and to enhance discretion.

In an aspect of the disclosure, the pad comprises a welded edge.

The term welded edge denotes: an edge cut by welding or by ultrasonic cutting. The technical effect of the welding makes it possible to render the pad impervious. The welding or ultrasonic cutting makes it possible to avoid stitching in the fabric and enhances the imperviousness as well as the isolating properties of the textile. It also prevents fraying as the fibres are fused at the level of the cut area. The cutting method is very rapid.

In an aspect of the disclosure, the pad comprises a rounded preformation having a spherical cavity positioned substantially at the centre of the pad on the inner face.

The nipples are rendered very sensitive by nursing. The preformation makes it possible to prevent irritations and friction.

In an aspect of the disclosure, the pad comprises edging forming a return on the edge of the pad.

The edging makes it possible to render the pad impervious on the side edge by preventing breast milk leakage via the edges. The edging is laminated, preventing risks of breast milk leakage via the needle holes. The edging is provided with a non-slip treatment making it possible to prevent the pad from moving in the bra cup.

In an aspect of the disclosure, the edging is composed of a strip covering a part of the top face and of the bottom face of the pad.

In an aspect of the disclosure, the edging comprises a coating, of silicone or polyurethane type, to apply a non-slip effect.

In an aspect of the disclosure, the pad comprises a coating, of silicone or polyurethane type. In an example, the coating is a pattern applied to the pad.

According to a second aspect, the present disclosure relates to a method for manufacturing a washable multilayer nursing pad, it comprises:

a) a lamination step using the following successive layers:
a knitted mesh layer,
a non-woven fibre layer,
a microporous layer, and
a protective layer,
   said lamination step forms a multilayer assembly.

In an aspect of the disclosure, the method also comprises the following step:

b) a step of preforming of the pad from the multilayer assembly formed in step a), said step of preforming is configured to render the multilayer assembly convex;

In an aspect of the disclosure, the method comprises a subsequent step, after step b): a step c) of welding of the edge of the pad.

In an aspect of the disclosure, also comprising a subsequent step, after step c): a step d) of edging by thermofusing of the pad forming an extra thickness on the edge of the pad.

The lamination step makes it possible to observe the technical features of the materials and preserve the breathability of the pad. Lamination makes it possible to assemble the four layers in a single layer.

The preforming step gives the pad the shape of the breast and the cavity for the preformation of the nipple.

The finishing carried out in this step provides the pad with the possibility of isolating breast milk and preventing any leakage via the edges but also the finishing of the edges.

The advantages, aims and particular features of this method for manufacturing a washable nursing pad being similar to those of the washable multilayer nursing pad according to the present disclosure, they are not reiterated herein.

### BRIEF DESCRIPTION OF THE DRAWINGS

Further advantages, aims and features of the present disclosure emerge from the following description, provided, for explanatory and in no way limitative purposes, with reference to the appended drawings, wherein:

FIG. 1, represents an exploded view of an aspect of the pad comprising four layers of the nursing pad.

FIG. 2 represents an external view of the pad.

FIG. 3 represents an internal view of the pad.

FIG. 4 represents a side view of the pad.

FIG. 5 represents a schematic side view of the pad in a cross-section along a longitudinal axis A-A.

FIG. 6 represents the steps of manufacturing the pad.

### DETAILED DESCRIPTION

In an aspect of the disclosure, the washable nursing pad comprises three successive layers. FIG. 1 represents a further aspect of the disclosure wherein the nursing pad comprises four successive layers. FIG. 1 represents an exploded view of the four layers of the nursing pad.

FIG. 1 comprises a 3D knitted mesh layer 20, a non-woven fibre layer 21, a microporous layer 22, a protective layer 23, an antibacterial polyester layer 24, a monofilament layer 25 and a polyester layer 26.

The first layer is a 3D knitted mesh layer 20 made of three dimensions by knitting three different materials. The 3D knitted mesh layer 20 comprises of a bottom face comprising an antibacterial polyester layer 24. The antibacterial polyester layer 24 is in direct contact with the skin. The structure of the antibacterial polyester layer 24 is perforated. The perforated structure of the antibacterial polyester layer 24 allows breast milk to be evacuated in the upper layers. In an aspect of the disclosure, the antibacterial polyester layer 24 has a mass of at least 75 g/m2. In a further aspect of the disclosure, the antibacterial polyester layer 24 has a mass of 100 g/m2. The antibacterial polyester layer, to obtain the antibacterial feature thereof, undergoes a specific treatment. There are different types of antibacterial treatments for textiles. For example, it is possible to use the finishing technique wherein the textile is immersed in a solution comprising an active substance and a binder. The binder enables the active substance to remain bonded to the textile. The 3D knitted mesh 20 is composed of a top face comprising a polyester layer 26. The structure of the polyester layer 26 is structured so as to transfer the liquid to the upper layers. In an aspect of the disclosure, the polyester layer 26 has a mass of at least 100 g/m2. In a further aspect of the disclosure, the polyester layer 26 has a mass of 135 g/m2. The core of the knitted mesh layer 20 is composed of a monofilament layer 25 made of polyester. A monofilament is an extra-long single textile filament, obtained in the manufacture of artificial or synthetic textiles giving this textile a very aerated structure. In an aspect of the disclosure, the monofilament layer 25 has a mass of at least 70 g/m2. In a further aspect of the disclosure, the monofilament layer 25 has a mass of 95 g. The monofilament layer 25 is knitted between the antibacterial polyester layer 24 and the polyester layer 26.

The second layer is a non-woven fibre layer 21 positioned above the top face of the 3D knitted mesh layer 20. The non-woven fibre layer 21 is composed for example of 50% polyester and 50% viscose. The non-woven fibre layer 21 is obtained by processing the fibres using a mechanical needle system. The density of the non-woven fibre layer 21 is at least 75 g/mm of thickness. In an aspect of the disclosure, the density of the non-woven fibre layer 21 is 100 g/mm of thickness. The non-woven fibre layer 21 absorbs for example at least 5 L/m² of breast milk and at least 8 L/m² of water. In an aspect of the disclosure, the non-woven fibre layer 21 absorbs 6.45 L/m² of breast milk and 9.27 L/m² of water. The non-woven fibre layer 21 is positioned above the 3D knitted mesh layer 20.

The third layer is a microporous layer 22. The microporous layer 22 is impervious and breathable. The microporous layer 22 is composed in an aspect of the disclosure of 60% polyester and 40% polyurethane. In a further aspect of the disclosure, the microporous layer is composed of 100% polyester. The structure of the microporous layer 22 gives it the breathability feature while retaining breast milk. The microporous layer 22 gives the nursing pad the imperviousness feature thereof. The microporous layer 22 is positioned above the non-woven fibre layer 21.

The fourth layer is a protective layer 23. The structure of the protective veil is light and aerated while providing the protective layer 23 with the feature of preventing snagging and scratching. The protective layer 23 is positioned above the microporous layer 22. The protective layer 23 is situated opposite the 3D knitted mesh layer 20.

FIG. 2 represents an external view of the pad. FIG. 2 comprises the protective layer 23 and the edging 27 by a thermofused strip. The pad has an equilateral triangle shape. The pad has a shape comprising rounded angles. The edges of the pad are slightly rounded.

FIG. 3 represents an internal view of the pad. FIG. 3 comprises an antibacterial polyester layer 24, the edging 27 by a thermofused strip and the cavity of the preformed nipple 28. The preformed nipple recess 28 is formed to reduce sensations of irritation and discomfort. FIG. 3 has a longitudinal axis A-A used hereinafter for FIG. 5.

FIG. 4 represents a side view of the pad. FIG. 4 comprises the antibacterial polyester layer 24, the protective layer 23 and the edging 27 by a thermofused strip. The nursing pad is curved so as to mould the shape of the breast.

FIG. 5 represents a schematic side view of the pad in a longitudinal cross-section along an axis A-A. FIG. 5 comprises the non-woven fibre layer 21, the 3D knitted mesh layer 20 and the cavity of the preformed nipple 28. The depth of the preformed nipple recess 28 is between 0.1 and 0.5 cm. In an aspect of the disclosure, the depth of the preformed nipple recess 28 is 0.3 cm. In the thickest part thereof, the pad has a thickness between 0.2 and 0.9 cm. In an aspect of the disclosure, the thickness of the pad is 0.8 cm. The diameter of the preformed nipple recess 28 is at least 1 cm. In an aspect of the disclosure, the diameter of the recess of the preformed nipple 28 is 1.5 cm.

FIG. 6 represents an aspect of the washable nursing pad.

The first step consists of lamination 29. Lamination 29 is an assembly of material by bonding processes. In an aspect of the disclosure, the process of lamination 29 is carried out using the following successive layers:
a knitted mesh layer (20),
a non-woven fibre layer (21),
a microporous layer (22), and
a protective layer (23).

The process of lamination 29 forms a multilayer assembly. The process of lamination 29 observes the technical features of the different materials forming the successive layers as well as the breathability of the pad.

The second step consists of a preforming 30 of the pad. The preforming 30 is carried out using the multilayer assembly formed by the process of lamination 29. Preforming 30 consists of giving the pad a rounded shape moulding the shape of the breast. The preforming 30 provides a rounded shape having a spherical cavity positioned substantially at the centre of the pad on the inner face creating a recess for the nipple.

The third step consists of edging by thermofusing 31. The edging by thermofusing 31 is there to form the edge of the pad. The edge is defined by a side edge and a part of the top face and a part of the bottom face of the pad. The edge of the pad is represented by a strip. The edging 27 is composed of a strip of at least 7 mm covering by 3 mm each side of the pad and by 1 mm the side edge of the pad. In an aspect of the disclosure, the edging 27 comprises a strip of 16 mm covering by 7 mm each side of the pad and by 2 mm the side edge of the pad. The strip used for thermofusing benefits from a non-slip finish.

### LIST OF REFERENCES

20 Knitted mesh layer

21 Non-woven fibre layer

22 Microporous layer

23 Protective layer

24 Antibacterial polyester layer

25 Monofilament layer

26 Polyester layer

27 Edging

28 Preformed nipple recess

29 Lamination

30 Preforming

31 Edging by thermofusing

What is claimed is:

1. A washable multilayer nursing pad, comprising an edge and successive layers:
a knitted mesh layer at least 1.5 mm thick and perforated, comprising a top face in direct contact with the skin and a bottom face;
a non-woven fibre layer, in contact with the top face, capable of absorbing at least 2.5 L/m² of water;
a microporous layer comprising an impermeable and breathable structure; and
a protective layer covering the entire microporous layer.

2. The pad according to claim 1, wherein the knitted mesh layer comprises:
the top face composed of a first textile fibre layer in direct contact with the skin, said first textile fibre layer is composed of a plurality of perforating openings on an outer surface;
the bottom face composed of a second textile fibre layer in contact with the non-woven fibre layer; and
a monofilament layer at least 1.5 mm thick composed of textile fibre, inserted between the first textile fibre layer and the second textile fibre layer.

3. The pad according to claim 2, wherein the first textile fibre layer is made of antibacterial polyester and the second fibre layer is made of polyester.

4. Pad The pad according to claim 1, wherein the non-woven fibre layer has a thickness between 2.5 mm and 8 mm, preferably between 5 mm and 7 mm.

5. The pad according to claim 1, wherein the pad comprises a substantially triangular shape with rounded angles.

6. The pad according to claim 1, wherein the pad comprises an ergonomic rounded preformation to mould the shape of the breast.

7. The pad according to claim 1, wherein the pad comprises a welded edge.

8. The pad according to claim 1, wherein the pad comprises a rounded preformation having a spherical cavity positioned substantially at the centre of the pad on the inner face.

9. The pad according to claim 1, wherein the pad comprises edging forming a return on the edge of the pad, said edging is composed of an impervious strip covering a part of the top face and of the bottom face of the pad and forming an extra thickness on the edge of the pad.

10. The pad according to claim 9, wherein the edging comprises a coating, of silicone or polyurethane type, to apply a non-slip effect.

11. The pad according to claim 1, wherein the pad comprises a coating, of silicone or polyurethane type.

12. A method for manufacturing a washable multilayer nursing pad according to claim 1, wherein the method comprises:
a) a step of lamination using the following successive layers:
a knitted mesh layer,
a non-woven fibre layer,
a microporous layer, and
a protective layer,
said step of lamination forms a multilayer assembly.

13. The method according to claim 12 also comprising a subsequent step, after step a):
b) a step of preforming of the pad from the multilayer assembly formed in step a), said step of preforming is configured to render the multilayer assembly convex.

14. The method according to claim 13 also comprising a subsequent step, after step b):
c) a step of welding of the edge of the pad.

15. The method according to claim 14 also comprising a subsequent step, after step c):
d) a step of edging by thermofusing of the pad forming an impervious extra thickness on the edge of the pad.
